(19) United States
(12) Patent Application Publication
(10) Pub. No.: US 2014/0066363 A1
(43) Pub. Date: Mar. 6, 2014

(54) CARBOHYDRATE NANOPARTICLES FOR PROLONGED EFFICACY OF ANTIMICROBIAL PEPTIDE

(71) Applicants: Arun K. Bhunia, West LaFayette, IN (US); Yuan Yao, West LaFayette, IN (US)

(72) Inventors: Arun K. Bhunia, West LaFayette, IN (US); Yuan Yao, West LaFayette, IN (US)

(21) Appl. No.: 13/983,585

(22) PCT Filed: Feb. 6, 2012
(86) PCT No.: PCT/US2012/023919

§ 371 Date: Nov. 18, 2013

Related U.S. Application Data
(60) Provisional application No. 61/440,238, filed on Feb. 7, 2011.

(51) Int. Cl. A61K 38/16

(52) U.S. Cl. CPC A61K 38/164
USPC 514/2.4

(57) ABSTRACT

A nanoparticle includes a carbohydrate carrier and a bacteriocin. A method for prolonging efficacy of a bacteriocin against a food pathogen includes providing the bacteriocin in a delivery system, and inhibiting the food pathogen by the bacteriocin. A duration of efficacy of the bacteriocin against the food pathogen when the bacteriocin is provided in the delivery system exceeds a duration of efficacy of the bacteriocin when the bacteriocin is provided without the delivery system.

### PRIORITY

This application claims the benefit of priority under 35. U.S.C. 119(e) to U.S. Provisional Patent Application No. 61/440,228, filed Feb. 7, 2011, which is incorporated by reference herein in its entirety.

### FEDERALLY SPONSORED RESEARCH OR DEVELOPMENT

This invention was made with government support under Grant No. CBET0932586 awarded by the National Science Foundation and Grant No. 2009-35603-05004 awarded by the United States Department of Agriculture. The government has certain rights in the invention.

### TECHNICAL FIELD

The present teachings relate generally to nanoparticles and, more particularly, to carbohydrate nanoparticles for prolonging the release of antimicrobial peptides in food systems.

### INTRODUCTION

Successful control of food pathogens not only saves human lives but also has profound national and international economic implications. Although traditional prevention and intervention methodologies are important, the need for additional novel concepts and technologies persists.

Nisin is an amphiphilic, membrane pore-forming bacteriocin and FDA-approved food-grade antimicrobial peptide which, like other antimicrobial compounds, is effective in inhibiting pathogenic bacteria in food and other nutrient-containing systems. However, these compounds are often subjected to rapid depletion after initial application and lose their antimicrobial activities very quickly. The depletion is believed to be caused by physical diffusion or adsorption and/or by chemical degradation (Delves-Broughton, 2005; Quintavalla and Vicini, 2002; Rose et al., 1999). In addition, free nisin added to a food surface may diffuse to the bulk of food, reducing its capability to inhibit bacteria growth at the surface.

To prolong its efficacy, nisin has been incorporated into packaging films and coatings (Joerger, 2007; Neetoo et al., 2007; Padgett, et al., 1998; Quintavalla and Vicini, 2002; Siragusa et al., 1999). The challenges of active packaging, however, lie in the high cost of producing films on an industrial scale and in tailoring peptide release. Recently, liposome-encapsulated nisin was tested in milk fermentation (Laridi et al., 2003) and in the ripening of Lactobacillus-containing cheddar cheese (Benech et al., 2003). The stability and entrapment of nisin in liposomes has been studied (Taylor et al., 2007; Were et al., 2003), and a remaining hurdle for a liposome strategy is to achieve controlled release.

### SUMMARY

The scope of the present invention is defined solely by the appended claims, and is not affected to any degree by the statements within this summary.

By way of introduction, a nanoparticle embodying features of the present teachings includes a carbohydrate carrier and a bacteriocin.

A method for prolonging efficacy of a bacteriocin against a food pathogen embodying features of the present teachings includes providing the bacteriocin in a delivery system, and inhibiting the food pathogen by the bacteriocin. A duration of efficacy of the bacteriocin against the food pathogen when the bacteriocin is provided in the delivery system exceeds a duration of efficacy of the bacteriocin when the bacteriocin is provided without the delivery system.

### BRIEF DESCRIPTION OF THE DRAWINGS

FIG. 1 shows a BHI-agar deep-well model of peptide depletion during storage (left) and activity bioassay against a pathogen (right). The model and bioassay are related through aliquot transfer (dotted lines).

FIG. 2 shows a chain length distribution of phytoglycogen (PG) and phytoglycogen β-dextrin (PGB).

FIG. 3 shows TEM images of phytoglycogen (PG), phytoglycogen β-dextrin (PGB), phytoglycogen octenyl succinate with DS 0.12 (PG-OS (0.12)), and phytoglycogen β-dextrin octenyl succinate with DS 0.119 (PGB-OS (0.12)). Scale bar: 100 nm.

FIGS. 4A and 4B show a schematic of phytoglycogen (FIG. 4A) and phytoglycogen β-dextrin (FIG. 4B) nanoparticles. Beta-amylolysis occurs at the surface of phytoglycogen nanoparticle, removing a certain amount of maltosyl units from long external chains to yield phytoglycogen β-dextrin. In FIG. 4B, the red circles highlight the branch units at the surface of the nanoparticle. These branch units are nearly intact, which maintains the particle size of the nanoparticle after 6-amylolysis.

FIG. 5 shows zeta-potential (pH 5.5) of phytoglycogen derivatives with and without nisin. Mean values are shown with error bars of standard deviations (n=3).

FIG. 6 shows amount of non-loaded nisin indicated by nisin concentrations of the filtrates collected from the centrifugal ultrafiltration of preparations containing 20 μg/mL nisin. Mean values are shown with error bars of standard deviations (n=3).

FIG. 7 shows correlation between the amount of free nisin and the size of inhibitory ring against L. monocytogenes in bioassay.

FIG. 8 shows the initial availability of nisin for nanoparticle solutions containing 100 μg/mL nisin. Mean values are shown with error bar of standard deviations (n=3).

FIG. 9 shows inhibitory rings of the solution of free nisin and nisin preparations containing PG-OS (0.12) or PGB-OS (0.12) at the initial stage (0 day) and at 7 and 15 days of storage at 4° C. For groups at 7 and 15 days, to the left of the vertical dotted line are inhibitory rings of aliquots transferred from the BHI-agar gel deep wells (labeled as “deep-well model”), and to the right of the dotted line are inhibitory rings of nisin preparations stored in regular test tubes (labeled as “reference”).

FIGS. 10A-B show retention of nisin activity during the 21-day 4° C. storage in the BHI-agar deep-well model for phytoglycogen-based (FIG. 10A) and phytoglycogen β-dextrin-based (FIG. 10B) derivatives. Nisin activity was quantified using the size of inhibitory ring. Mean values are shown with error bar of standard deviations (n=3).

FIG. 11 shows a schematic of using carbohydrate nanoparticle-stabilized emulsions to prolong the efficacy of nisin. The same initial amount of nisin is in a solution of free molecules (left column) or in an emulsion (right column). Oil droplets in the emulsion are stabilized by amphiphilic carbohydrate nanoparticles (PG-OS) (yellow spheres). In each system, the distribution of nisin is depicted at two stages: freshly prepared and after extended storage. The grey area indicates nisin depletion factors, including diffusion, irreversible adsorption, and chemical degradation.

FIG. 12 shows transmission electron microcopy (TEM) of PG-OS and WCS-OS (scale bar: 200 nm).

FIG. 13 shows a light-scattering intensity-based distribution of hydrodynamic diameters of PG-OS nanoparticles and a PG-OS-stabilized emulsion (A), and of WCS-OS molecules and a WCS-OS-stabilized emulsion (B). Each emulsion contained 150 μg/mL nisin. Original: PG-OS or WCS-OS solution before homogenization. Homogenized: PG-OS or WCS-OS solution after homogenization. Emulsion: emulsion stabilized using PG-OS or WCSOS.

FIG. 14 shows the impact of nisin concentration on the zeta-potential of emulsions stabilized using PG-OS, WCS-OS, and Tween 20. Mean values are shown with standard deviations as error bars (n=3).

FIG. 15 shows images of the preservation of nisin activity against L. monocytogenes by various delivery systems during 4° C. storage. The label “free nisin” denotes the preparation containing nisin only in buffer. The total initial concentration of nisin was 150 μg/mL for each preparation. One portion of each preparation was applied to the model system (labeled “Model”), and another portion was stored in a regular test tube as the control (labeled “Control”). Both model and control groups were aliquoted after various storage periods for the activity tests.

FIGS. 16A-C show preservation of nisin activity against L. monocytogenes during a 50-day storage period at 4° C. in the BHI-agar deepwell model with preparations containing 150 μg/mL (FIG. 16A) or 200 μg/mL (FIG. 16B) nisin, as well as a 6-order polynomial “standard curve” (FIG. 16C) of inhibitory ring size vs. the amount of available nisin. In A and B, the label “free nisin” denotes the preparation containing nisin only in buffer, and the labels “PG-OS,” “WCS-OS,” and “Tween 20” denote preparations in which nisin was incorporated within the emulsions stabilized by these emulsifiers. Mean values are shown with error bars indicating standard deviations (most error bars are indiscernible due to low values) (n=3).

### DETAILED DESCRIPTION

As further described herein, soluble nanocarriers can reduce the depletion of active compounds during storage without sacrificing their availability in times of need (i.e. in the presence of pathogenic contamination). A number of colloidal assemblies have been explored, such as polymersomes, particle-stabilized emulsions and colloidosomes, and layer-bylayer microcapsules. Over a century ago, Pickering (1907) indicated that colloidal particles could be used to stabilize emulsions, forming so-called “Pickering emulsions.” Recently, there has been a resurgence of interest in micro- and nanoparticle-stabilized emulsions, mostly due to the use of the interfaces as templates for nano-construction. The distinct properties of these emulsions are attributable to the very large free energy of adsorption of the particles, which usually leads to highly stable emulsions (Aveyard et al., 2003).

Associated with particle-stabilized emulsions, the concept of “colloidosomes” was proposed by Velev et al. (1996) (the term was coined by Dinsmore et al. (2002)) as selectively permeable capsules composed of colloidal particles. The colloidal particles used in emulsions thus far have been either inorganic or synthetic polymer-based “hard” particles such as silica particles and barium sulfate, calcium carbonate, bentonite, polystyrene, polytetrafluoroethylene (Aveyard et al., 2003; Binks et al., 2007), and Au-, Ag-, or Fe₃O₄-based nanoparticles (Wang et al., 2005). Due to the necessity of cross-linking individual particles for colloidosomes, synthetic materials such as polystyrene derivatives, poly-(divinylbenzene-alt-maleic anhydride), and poly-(methylmethacrylate) have also been used (Rossier-Miranda et al., 2009). However, it would likely be difficult to use these materials to construct colloidal assemblies for orally delivered systems such as food, nutraceuticals, and drugs.

The present inventors have discovered that carbohydrate nanoparticles can prolong the efficacy of antimicrobial peptides against pathogens, and describe a novel methodology for improved food safety that allows controlled delivery of a broad variety of bioactive compounds. In addition, the present inventors have discovered that the use of all manner of emulsions (e.g., emulsions stabilized by PG-OS, WCS-OS, modified starch, gum arabic, whey protein and casein, phospholipids, and the like)—particularly emulsions with a negative charge at the interface (i.e. the surface of oil droplets)—is effective. Without wishing to be bound by a particular theory or to in any way limit the scope of the appended claims or their equivalents, it is presently believed that this negative charge at the surface of oil droplets is used to interact with positively charged bacteriocin, such as nisin.

In some embodiments, the present inventors prepared an amphiphilic carbohydrate nanoparticle, phytoglycogen octenyl succinate (PG-OS), and used PG-OS-stabilized emulsion to deliver functional peptides with prolonged efficacy. PG-OS was prepared through octenyl succinate (OS) substitution (an FDA-approved reaction for food usages) of phytoglycogen (PG), a major carbohydrate nanoparticle in the su1-containing plants such as maize.

Throughout this description and the appended claims, the phrase “phytoglycogen or glycogen-type material” refers to dendritic (i.e., highly branched) α-D-glucan and carbohydrate nanoparticles. The term “phytoglycogen” generally refers to material that is derived from plants while the term “glycogen” generally refers to material that is derived from microbials and/or animals.

In previous studies, the present inventors have shown that PG-OS is partially digestible and can form emulsions with outstanding physical and oxidative stability (Scheffler et al., 2010a, b). In the current study, the PG-OS interfacial layer was used to adsorb nisin through electrostatic and hydrophobic interactions for extended efficacy against Listeria monocytogenes.

In some embodiments, the nanocarriers used in accordance with the present teachings are negatively charged, phytoglycogen-based dendritic polysaccharides that adsorb positively charged nisin molecules via electrostatic interactions. Phytoglycogen (PG) was isolated from mutant maize, followed by an enzymatic modification and succinylation or octenyl succinylation.

For nisin binding, phytoglycogen octenyl succinate (PG-OSA) and phytoglycogen succinate (PG-SA) were dissolved in 0.05 M pH 5.5 NaAc buffer at 0.5% and added with 0.1% of nisin. After 24 hr incubation, the mixture was evaluated for the physical binding of nisin and the anti-listerial activity profile in a model BHI-agar system. The results indicated effective PG modifications, substantial nisin binding, and prolonged release of anti-listerial activity. TEM images showed that PG particles ranged from 30 to 100 nm. Zeta-potential of substituted PG reached up to −39.5 mV. Ultrafiltration assay showed that up to 76% of nisin was bonded to PG derivatives. When nisin preparations were stored in the deep wells in the BHI-agar gel, free nisin displayed the quickest reduction of activity and the retained activity was negligible at day 5. In contrast, binding with PG derivatives prolonged nisin activity to up to 15 days. In addition, the type and the degree of substitution of PG derivatives affected the binding and release properties of nisin. Overall, enzymatic modification was beneficial to improved binding affinity and prolonged release, and the PG-OSA nanocarriers were superior compared with PG-SA. The methodology developed by the present inventors has the potential to prolong the inhibition effect of nisin on the growth of Listeria monocytogenes on the surface of foods, such as deli meats. PG-based nanocarriers may have unique benefits for the safety and quality of food.

In principle, amphiphilic nisin molecules can be enriched at the oilwater interface and protected by the emulsifier layer from a quick depletion. Without wishing to be bound by a particular theory or to in any way limit the scope of the appended claims or their equivalents, it is presently believed that that negatively charged emulsifiers are superior to neutral emulsifiers to retain nisin (positively charged) against L. monocytogenes. Waxy corn starch octenyl succinate (WCS-OS) and phytoglycogen octenyl succinate (PG-OS) were used as models of negatively charged emulsifiers, and Tween 20 was used as a model of neutral emulsifier. WCS-OS, PG-OS, and Tween 20 were dispersed in buffer and added with oil. The mixtures were subjected to homogenization and thereafter added with the same amount of nisin. To evaluate the depletion of nisin activity, each preparation was added to BHI-agar wells, aliquoted after various storage periods, and measured for the retention of inhibitory activity against L. monocytogenes. The preliminary data indicated that the retention of nisin activity was much higher in PG-OS and WCS-OS-stabilized emulsions than in the free nisin dispersion and Tween 20-stabilized emulsion.

Thus, in some embodiments, nisin and Listeria monocytogenes were used as the peptide and pathogen models, respectively, and phytoglycogen (PG)-based nanoparticles were developed as carriers of nisin. PG from su1 mutant maize was subjected to β-amylolysis as well as subsequent succinate or octenyl succinate substitutions. The goal was to minimize the loss of peptide during storage and meanwhile realize an effective release in the presence of bacteria. The capabilities of PG derivatives as carriers of nisin were evaluated using centrifugal ultrafiltration, zeta-potential, and the initial availability of nisin against L. monocytogenes. All methods indicated that nisin loading was favored by a high degree of substitution (DS), presence of hydrophobic octenyl moiety, and β-amylolysis of PG nanoparticles. To evaluate the prolonged nisin efficacy, preparations containing nisin and PG derivatives were loaded into a BHI-agar deep-well model (mimicking nisin depletion at the nutrient-containing surface). The residual inhibitory activities of preparations against L. monocytogenes were monitored during 21 days of storage at 4° C. The results showed that all PG derivatives led to the prolonged retention of nisin activity and the longest retention was associated with high DS, β-amylolysis, and octenyl succinate. Evidently, both electrostatic and hydrophobic interactions are the driving forces of nisin adsorption, and the glucan structure at the nanoparticle surface also affects nisin loading and retention during storage.

L. monocytogenes is a gram-positive food borne microorganism [1] that grows widely in environments, even at refrigerated temperatures, and survives for a long period of time in manufacturing plants and on food surfaces. It is responsible for outbreaks and a number of recent USDA recalls [2, 3]. According to the Center for Disease Control and Prevention (CDC), listeriosis is a serious infection and an important public health problem. Listeriosis causes hundreds of deaths each year in the U.S. and there is zero tolerance policy for L. monocytogenes in ready-to-eat foods. An effective strategy to reduce the risk of listeriosis will have a profound impact on society and may help save lives.

Nisin is produced from Lactococcus lactis fermentation. It is a positively charged lantibiotic peptide [4-7] that is able to bind to negatively charged cytoplasmic membranes. Nisin contains 34 amino acids and has a molecular weight of 3.4 kD. It has been approved as a food preservative and is effective in suppressing Gram-positive bacteria such as L. monocytogenes. Nisin kills bacteria by forming pores on cell membranes [8] and can be used broadly in food [9].

The antibacterial efficacy of nisin during storage is governed by multiple factors. Migration of nisin to a food mass reduces its effect at the food surface [10]. Components such as proteases and glutathione [11], titanium dioxide, and sodium metabisulphite can adversely affect nisin stability [9]. In order to prolong its efficacy, nisin has been incorporated in packaging films or coatings [10, 12-15]. The challenges for this strategy lie in the cost of film-making on an industrial scale and in the tailoring of nisin release. Recently, liposome-encapsulated nisin has been constructed and tested in milk fermentation [16] and the ripening of Cheddar cheese [17]. The stability and entrapment efficiency of nisin in liposome has also been studied [18, 19].

Phytoglycogen (PG) is a water-soluble glycogen-like α-D glucan in plants. The largest source of PG is the maize mutant su1, a major genotype of sweet corn. The su1 mutation leads to a deficiency in SU1, an isoamylasetype starch debranching enzyme (DBE) [20]. In amyloplasts, starch synthases, branching enzymes, and DBE work in concert to synthesize starch [21]. The role of DBE is to trim abnormal branches that inhibit the formation of starch granules [22, 23]. In the absence of DBE, the highly branched PG is formed to replace starch.

Chemical modifications have been used to bring functionalities to PG nanoparticles [24, 25]. Among food-related reactions, succinate substitution is used to bring negative charges, and octenyl succinate substitution is used to bring negative charges and hydrophobicity [26]. For both, the properties of PG derivatives can be controlled by the degree of substitution.

In this study, PG was subjected to β-amylolysis and subsequent succinate or octenyl succinate substitution. PG derivatives were evaluated for their capability for loading nisin and prolonging nisin efficacy against L. monocytogenes. The goal was to minimize the loss of peptide during storage and meanwhile realize an effective release in the presence of bacteria. The objective was to reveal the relationship between the structure of PG-based nanoparticles and prolonged antimicrobial efficacy. By this work, novel carbohydrate nanomaterials for enhanced performance of bioactive peptides for food were discovered.

Due to their biodegradability and functionality, the carbohydrate nanoparticles studied in this work may also contribute to the delivery of therapeutic proteins and peptides. In general, the efficacies of protein therapeutics are limited by their instability, immunogenicity, and shorter half lives [27]. To address these issues, a number of delivery systems have been designed, including covalent attachment of polyethylene glycol (and other biodegradable polymers) and adsorption or encapsulation with colloidal systems [27-34]. Recently, poly(lactic-co-glycolic acid) microspheres containing base or divalent cations were used as adjuvant of vaccines or to maintain the stability of encapsulated peptides [28, 29], and poly(lactic acid)-polyethylene glycol microspheres were used to deliver insulin [30]. At the micro- to nano-scales, both liposome and solid lipid particulates have been used to deliver peptides [31, 32], and the peptide loading was affected by factors including the surface charge and hydrophobicity. At the nano-scale, the Medusa system was commercially designed for delivering proteins and peptides [33]. This system consists of a poly L-glutamate backbone grafted with α-tocopherol, and the sustained drug release is based on reversible drug interactions with hydrophobic nanodomains of the nanoparticles [33]. Recently, amphiphilic copolymers of polylactic acid grafted onto hyperbranched polyglycerol were prepared to form a corona-core nanostructure and used to deliver protein [34]. Conceivably, the carbohydrate nanoparticles prepared in this study, such as negatively charged, amphiphilic phytoglycogen octenyl succinate, may have potential in the delivery of therapeutic proteins and peptides.

In some embodiments, an amphiphilic, negatively charged carbohydrate nanoparticle, phytoglycogen octenyl succinate (PG-OS), was used to form oil-in-water emulsion for delivering bacteriocin nisin against the food pathogen Listeria monocytogenes. Dynamic light scattering test showed that in emulsion, all PG-OS nanoparticles were adsorbed at the surface of oil droplets. Zeta-potential analysis indicated an effective adsorption of positively charged nisin molecules at the surface of PG-OS interfacial layer. Nisin depletion model showed that, during 50 days of storage, the anti-listerial activity of nisin-containing PG-OS-stabilized emulsion was substantially greater than that of nisin solution. In contrast, the emulsion stabilized with a neutral, small-molecule surfactant (Tween 20) or negatively charged, hyperbranched carbohydrate polymer (modified starch) was either ineffective or less effective than the nanoparticle-stabilized emulsion to retain nisin activity during storage.

The following examples and representative procedures illustrate features in accordance with the present teachings, and are provided solely by way of illustration. They are not intended to limit the scope of the appended claims or their equivalents.

Materials and Methods

Sweet corn Silver Queen (a su1 hybrid) was purchased from Burpee Co. (Warminster, Pa.). Bradford protein assay kit was purchased from Bio-Rad (Hercules, Calif.). Waxy corn starch was obtained from National Starch Food Innovation (Bridgewater, N.J.). Succinic anhydride, nisin, Tween 20, and isopropyl alcohol were purchased from Sigma-Aldrich (St. Louis, Mo.). 1-Octenyl succinic anhydride was obtained from Dixie Chemical Co. (Houston, Tex.). Beta-amylase, pullulanase, and isoamylase were purchased from Megazyme (Wicklow, Ireland). Brain Heart Infusion (BHI) and agar were purchased from BD (Franklin Lakes, N.J.).

Extraction of PG—Procedure A

Sweet corn kernels were ground into grits and then mixed with six weights of deionized water. The suspension was homogenized using a high-speed blender (Waring Laboratory, Torrington, Conn.) and then centrifuged at 8000 g for 20 min. The supernatant was collected and passed through a 270-mesh sieve. Three volumes of ethanol were added to the supernatant to precipitate polysaccharides. After centrifugation and decanting, the precipitate was suspended using ethanol and filtrated to dehydrate for three cycles. The solid material obtained after removing the residual ethanol was PG.

Extraction of PG—Procedure B

Sweet corn kernels were ground into grits and then mixed with four weights of deionized water. The suspension was homogenized using a high-speed blender (Waring Laboratory), and the solids were removed with a 270-mesh sieve. The liquid was adjusted to pH 4.8 to precipitate proteinaceous material. After centrifugation (10,000 g, 20 min), the supernatant was placed at 4° C. for 24 h and subjected to centrifugation (10,000 g, 20 min) to remove amylose. This procedure was repeated once. The collected supernatant was adjusted to pH 6.9, autoclaved (121° C., 20 min), and centrifuged (10,000 g, 20 min) after cooling. Three volumes of ethanol were added to the liquid collected to precipitate polysaccharides. The solid was further dehydrated with three cycles of ethanol dispersion-filtration and dried in a fume hood.

Preparation of PG β-Dextrin

Twenty grams of PG was dissolved in 400 mL pH 6.0, 50 mM sodium acetate buffer. One hundred microliters of β-amylase (1,800 U/mL) was added to the solution. The reaction was conducted in a shaking water bath (60° C., 70 rpm) for 10 h. Three volumes of ethanol were added to the reactant. After the centrifugation and decanting, the precipitate was suspended using ethanol and filtrated for three cycles. The solid obtained after removing the residual ethanol was PG β-dextrin (PGB).

Preparation of Non-Granular Starch

To prepare non-granular waxy corn starch (WCS), 20 g of WCS was dispersed in 400 mL of sodium hydroxide solution (2%, w/v) by heating in a boiling-water bath for 10 min. After cooling, the dispersion was adjusted to pH 7.0 using hydrogen chloride (10%, w/w). Three volumes of ethanol were added to the dispersion to precipitate polysaccharides. The solid was further dehydrated with three cycles of ethanol dispersion-filtration and dried in a fume hood.

Structure Analysis of PG and PGB

Weight-average molecular weight (M_{w}) and z-average root mean square radius (R_{z}) of PG and PGB were determined using the procedure described by Scheffler [24]. The chain length distribution of PG and PGB was characterized using the procedure described by Shin et al. [35].

Substitution of PG and PGB

Substitution of PG and PGB with octenyl succinate group was described by Scheffler et al. (24). Substitution with succinate group was essentially the same except that succinic anhydride was used in the replacement of 1-octenyl succinate anhydride. The materials collected were PG succinate (PG-S), PG octenyl succinate (PG-OS), PG β-dextrin succinate (PGB-S), and PG β-dextrin octenyl succinate (PGB-OS). Degree of substitution (DS) values PG-S, PG-OS, PGB-S, and PGB-OS materials were determined using a method described by Scheffler et al. [24]. For PG and WCS, the octenyl succinate (OS) substitution and determination of the degree of substitution (OS) were conducted as described by Scheffler et al. (2010a). The materials prepared were PG-OS and WCS-OS. TEM imaging and determination of molecular mass, root mean square (RMS) radius, and dispersed molecular density of both PG-OS and WCS-OS were conducted as described by Scheffler et al. (2010b).

Transmission Electron Microscopy (TEM)

TEM imaging of PG, PGB, and selected PG-OS and PGB-OS was conducted as described by Scheffler et al. [25].

Preparation of Nisin Solution

Commercial nisin solid contains 2.5% pure nisin, balanced with sodium chloride and denatured milk solids. To prepare nisin solution, 120 mg nisin solid was dissolved in 3.0 mL sodium acetate buffer (50 mM, pH 5.5), gently agitated for 15 h, and centrifuged at 5,000 g for 5 min at 15° C. The supernatant was collected as 1,000 μg/mL nisin solution.

Zeta-Potential Measurement for PG Derivatives

Zeta-potential was used to evaluate the surface charge density of the nanoparticles. To measure the zeta-potential, PG derivatives (1.0 mg/mL) were dissolved in sodium acetate buffer (50 mM, pH5.5) and loaded to Zetasizer Nano (Malvern, Westborough, Mass.) at room temperature. To evaluate the effect of nisin on the zeta-potential of nanoparticles, a 0.3 mL diluted nisin solution (200 μg/mL in sodium acetate buffer) was mixed with 2.7 mL solution of each PG derivative (1.0 mg/mL). After 24 h incubation at room temperature, the zeta-potential was measured for each mixture.

Nisin Loading to Nanoparticles

A centrifugal ultrafiltration device (Microsep, Pall Life Sciences) with molecular weight cut-off of 300 kD was used to evaluate the nisin loading to nanoparticles. In principle, non-loaded nisin molecules can pass through the membrane, whereas those loaded cannot. For the test, a 2.7 mL solution of PG or each of its derivatives (1.0 mg/mL) and 0.3 mL nisin solution (200 pg/mL), both in sodium acetate buffer (50 mM, pH5.5), were mixed and incubated for 30 min at room temperature. For each mixture, an aliquot of 2.7 mL was transferred to a Microsep tube and centrifuged (1,000 g at 15° C. for 2 h). From the filtrate, an aliquot of 800 μL was used to test the amount of nisin using the Bradford assay kit (Bio-Rad). Nisin solutions (2, 4, 8, 10, 12, 14, 16, 18, and 20 μg/mL) were used as the standards.

Preparation and Characterization of Nisin-Containing Emulsions

To prepare emulsions, PG-OS and WCS-OS were each dissolved in sodium acetate buffer (50 mM, pH 5.5, 22° C.) to form a solution of 10 mg/mL. As a reference, 1.0 mg/mL of Tween 20 solution was also prepared using the sodium acetate buffer. Vegetable oil was added to each emulsifier solution, at twice (for PG-OS and WCS-OS) or 20 times (Tween 20) the weight of the emulsifier. The mixtures were first subjected to high-speed homogenization (18,000 rpm for 1 min, T25 ULTRA-TURRAX, IKA) and then high-pressure homogenization (103 MPa, two cycles, Nano DeBee, BEE International). Subsequently, 4 mL of nisin solution (1500 or 2000 μg/mL) was added to a 16-mL-aliquot of collected emulsion. Each mixture was further diluted with the same volume of sodium acetate buffer. Using this procedure, emulsions were prepared to contain 150 (or 200) μg/mL nisin and 4.0 mg/mL PG-OS or WCS-OS or 0.40 mg/mL Tween 20. These emulsions were sterilized using a boiling-water bath for 3 min before further tests.

The distributions of particle size (denoted by hydrodynamic diameter) of the PG-OS and WCS-OS solutions (before and after homogenization at 103 MPa, two cycles) and the emulsions containing 150 μg/mL nisin were determined using a Zetasizer Nano (ZS90, Malvern Instruments) at 25° C. using the automatic setting with 1 min of equilibration. To determine zeta-potentials, emulsions containing 0, 150, and 200 μg/mL nisin were diluted to 20 volumes using 50 mM pH 5.5 sodium acetate buffer. The measurement was conducted at 25° C. using the automatic setting with 1 min of equilibration.

Bioassay of Nisin Inhibitory Activity Against L. Monocytogenes

Nisin activity, either for those freshly prepared or stored in BHI agar deep-well, was determined as described by Pongtharangkul and Demirci (2004) with modifications. Agar diffusion bioassay was used to determine the nisin activity against L. monocytogenes. BHI (3.7%) solution containing 0.75% agar and 1.0% Tween 20 was prepared and autoclaved. After cooling to approximately 40° C., the solution was inoculated by a 1.0% volume of BHI broth containing L. monocytogenes V7 (ca. 108 colony-forming units/mL). To each square Petri-dish plate (10×10 cm), a 32 mL inoculated BHI agar solution was added and allowed to solidify. Thereafter, holes with 7.0 mm diameter were made using a cork borer, and 100 μL of each nisin preparation was added to each agar well. The Petri-dish plates were then incubated for 24 h at room temperature, and the size of inhibitory ring was measured to indicate the activity against L. monocytogenes.

To correlate the nisin dose with its activity against L. monocytogenes, solutions with a series of nisin concentrations (20, 40, 60, 80, and 100 μg/mL) were prepared and subjected to the agar diffusion bioassay.

BHI-Agar Deep-Well Model to Evaluate Activity Depletion of Nisin

To prepare the BHI-agar deep-well model for nisin depletion test, a solution containing BHI (Brain Heart Infusion) solids (3.7%) and agar (1.0%) was autoclaved for 20 min at 121° C. The hot solution (225 mL) was poured into a 600-mL beaker to a height of 40 mm. After gel solidification, four wells (from gel surface to bottom) were made in each beaker using a 7.0-mm borer. Subsequently, 1.0 mL of nisin preparation was added to each well. Immediately after loading (day 0) and after 5, 10, 15, 20, 30, 40, and 50 days of storage at 4° C., a 100-μL aliquot of each nisin preparation was transferred from the well to a bioassay plate to determine the residual nisin activity.

Evaluation of Prolonged Nisin Efficacy

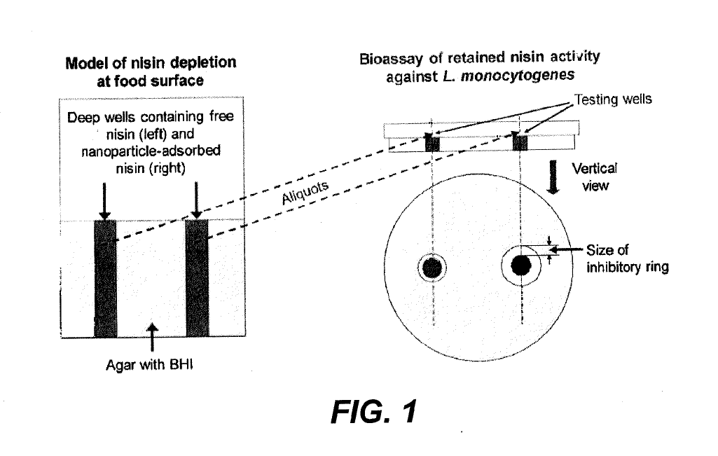

In this study, a BHI-agar deep-well model was established to mimic the depletion of nisin at nutrient-containing surfaces, such as a food surface (FIG. 1). BHI is a nutritious culture medium that supplies protein and other nutrients necessary to support the growth of fastidious and nonfastidious microorganisms. It contains infusions from calf brains and beef hearts, proteose and peptone, dextrose, sodium chloride, and disodium phosphate. In our earlier work, it was found that BHI-containing broth and gel always led to a rapid reduction or elimination of nisin activity, suggesting a nisin depletion effect. Therefore, BHI is an ideal nutrient model for studying nisin depletion and retention. Our experiments have consistently shown that BHI-containing broths and gels lead to rapid depletion of peptide activity. In a deep well filled with a liquid peptide preparation, peptide molecules diffuse from the solution into the gel (causing diffusion-based depletion), and BHI components diffuse from the agar gel into the solution (causing irreversible peptide adsorption or degradation).

For continuous sampling at various stages of storage, nisin preparations were added to the deep wells of the BHI-agar gel. For the deep-well storage, the inner surface of a well was used to mimic the outer surface of solid food. At the inner surface, nisin molecules diffuse from the solution toward the bulk of the gel, and BHI components diffuse from the agar gel to the solution. This process is comparable to what happens at the surface of gel-like foods applied with antimicrobial peptide: peptide molecules diffusing into food mass and food components diffusing to the aqueous layer at the surface.

To prepare BHI-agar gel with deep wells, a solution containing BHI solid (3.7%) and agar (1.0%) was autoclaved for 20 min at 121° C. The hot solution (225 mL) was poured into a 600-mL beaker to form a 40-mm height of liquid. After gel solidification, four wells (from gel surface to bottom) were made in each beaker using a 7.0-mm borer. To each well, a 1.0 mL nisin preparation containing 100 μg/mL nisin, either with or without PG derivatives (5.0 mg/mL), was added. Immediately after loading (0 day) and after 3, 5, 7, 10, 15, and 21 days of 4° C. storage, a 100 IJL aliquot of each nisin preparation was transferred from the well to the bioassay plate to determine the residual nisin activity against L. monocytogenes.

For each preparation, one portion was applied in the model and another portion was stored in a regular test tube as a reference. The references were used to evaluate the stability of nisin in the presence of nanoparticles only.

Structure of PG and PGB

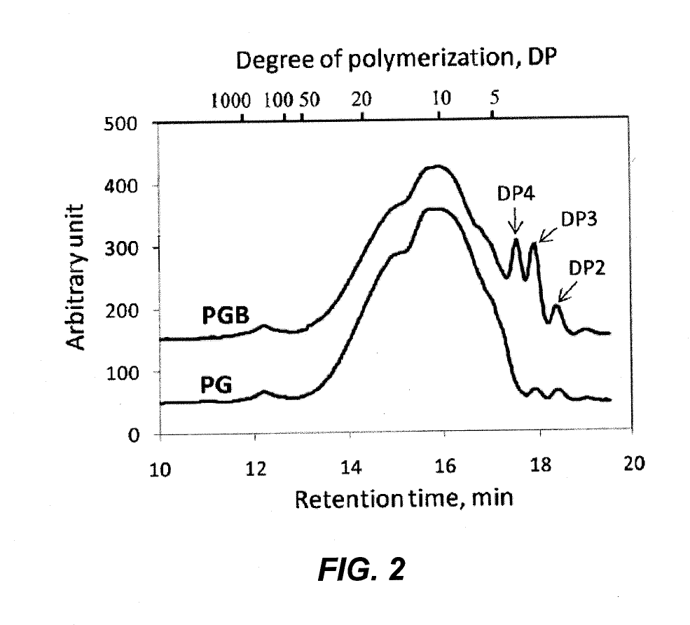

During β-amylolysis of α-D-glucan, two connected glucosyl units (i.e. one maltosyl unit) are continuously released from the non-reducing ends of external linear chains, producing β-dextrin. FIG. 2 shows the chain length distribution of PG and PGB. For PG, there is a large chain population at about DP 8-10 (DP: degree of polymerization) and a small one at about DP 16. There were also minor amounts of maltose (DP 2) and maltotriose (DP 3) but no maltotetraose (DP 4). For PGB, there was a substantial increase of DP 2, DP 3, and DP 4, suggesting the shortening of external chains due to β-amylolysis.

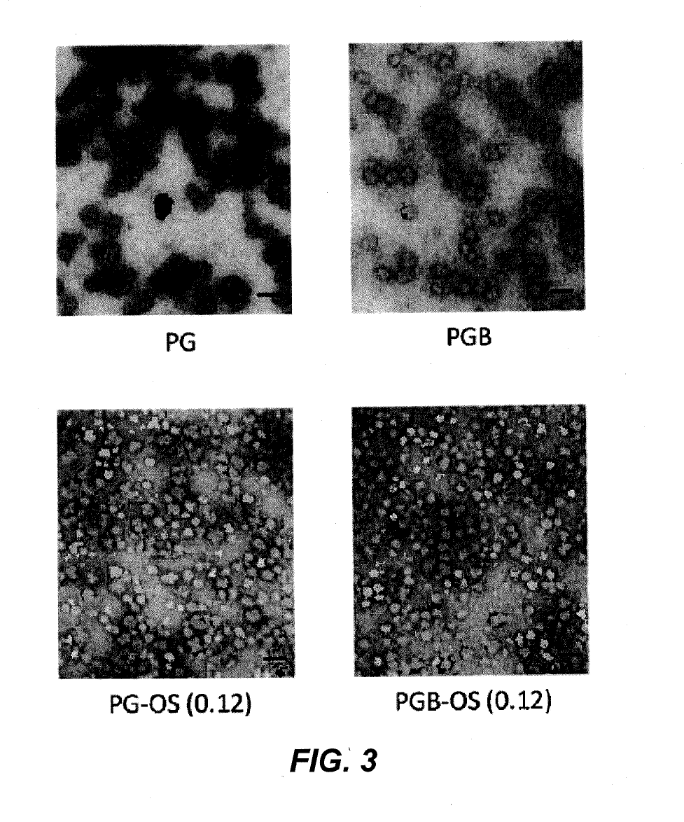

TEM images of PG and PGB indicate the presence of nanoparticles with sizes from 30-100 nm in diameter (FIG. 3). Most nanoparticles were 60-90 nm, which is comparable with the root mean square radius (Rz) of about 45 nm for both PG and PGB nanoparticles (Table 1).

TABLE 1
Weight-average molecular weight (M_{W}), z-average
root mean square radius (R_{Z}), and dispersed molecular
density (ρ) of phytoglycogen (PG) and phytoglycogen β-dextrin (PGB)

| | M_{W} × 10ₐ⁷, g/mol | R_{Z}, nm^{a} | ρ, (g/mol · nm³)^{ab} |
|---|---|---|---|
| PG | 7.9 ± 0.1 | 44.7 ± 0.6 | 885 ± 37 |
| PGB | 7.5 ± 0.3 | 45.2 ± 0.8 | 814 ± 47 |

^{a}Data are expressed in mean ± SD (n = 3)
^{b}ρ = M_{W}/(R_{Z})³

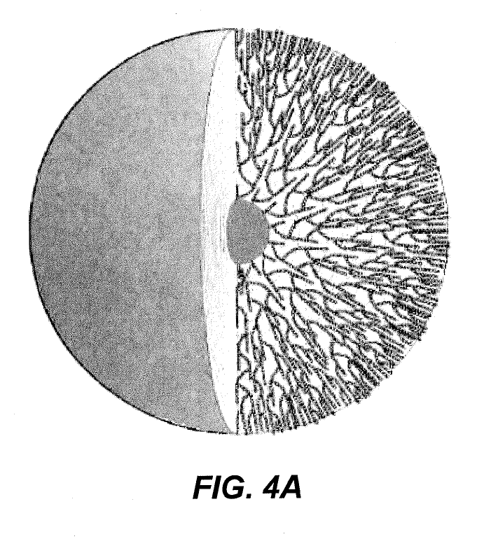

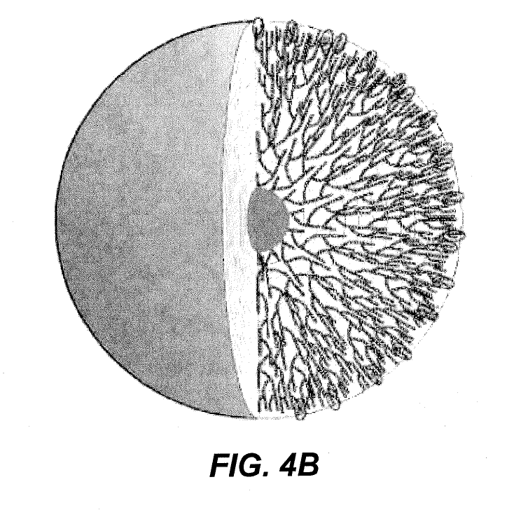

As shown in Table 1, the impact of β-amylolysis on the particle size (Rz) of PG was negligible, whereas the weight-average molecular weight (M_{w}) was slightly reduced from 7.9×10⁷ for PG to 7.5×10⁷ mol/g for PGB. FIGS. 4A-B depict the impact of β-amylolysis on PG structure. At the surface of a PG nanoparticle, there are long linear chains and newly formed branch units (FIG. 4A). After β-amylolysis, the long linear chains can be substantially shortened, whereas the branch units at the surface remained nearly intact (FIG. 4B). This resulted in essentially the same R_{z} value for PG and PGB. Due to β-amylolysis, M_{w} was reduced and the dispersed molecular density (p) was reduced accordingly. Conceivably, β-amylolysis had a thinning effect at the surface of nanoparticles, which would affect the loading capacity of modified nanoparticles (discussed later).

Succinate and Octenyl Succinate Substitution of PG and PGB

The degrees of substitution (OS) of PG derivatives are shown in Table 2. Evidently, higher doses of succinic anhydride (SA) or octenyl succinic anhydride (OSA) led to higher OS values. While the substitution efficiency was usually under 60%, the high doses (12% for SA and 26% for OSA) correlated with the high substitution efficiency (60%). Moreover, substrates (PG or PGB) and substitution reagents (SA and OSA) had negligible impact on the substitution efficiency. In this work, the following abbreviations are used to denote different PG and PGB derivatives: PG-S (0.05) and PG-S (0.12) for PG succinate with OS of 0.050 and 0.121 respectively, PGB-S (0.05) and PGB-S (0.12) for PGB succinate with OS of 0.050 and 0.120 respectively, PG-OS (0.05) and PG-OS (0.12) for PG octenyl succinate with OS of 0.049 and 0.120, respectively; and PGB-OS (0.05) and PGB-OS (0.12) for PGB octenyl succinate with OS of 0.048 and 0.119, respectively.

TABLE 2
Degree of substitution (DS) of PG and PGB subjected
to the reaction with succinic anhydride (SA)
or octenyl succinic anhydride (OSA)

| | | DS | | |
|---|---|---|---|---|
| | | | | Substitution |
| | | Theoretical | Measured^{a} | efficiency, %^{b} |
| | SA dose | | | |
| | (w/w glucan) | | | |
| PG | 6% | 0.10 | 0.050 ± 0.002 | 50 |
| | 12% | 0.20 | 0.121 ± 0.005 | 61 |
| PGB | 6% | 0.10 | 0.050 ± 0.001 | 50 |
| | 12% | 0.20 | 0.120 ± 0.003 | 60 |
| | OSA dose | | | |
| | (w/w glucan) | | | |
| PG | 13% | 0.10 | 0.049 ± 0.002 | 49 |
| | 26% | 0.20 | 0.120 ± 0.001 | 60 |
| PGB | 13% | 0.10 | 0.048 ± 0.003 | 48 |
| | 26% | 0.20 | 0.119 ± 0.005 | 60 |

^{a}Data are expressed in mean ± SD (n = 3)
^{b}Calculated as: measured DS/theoretical DS

TEM images of PG-OS (0.12) and PB-OS (0.12) are shown in FIG. 3. It appears that the particle sizes of both derivatives were a little smaller than those of PG and PGB. Compared with PG and PGB, there was less aggregation among the substituted nanoparticles possibly due to the electrostatic repulsion caused by the negative charges from substitution groups.

Zeta-Potential of PG Derivatives Affected by Nisin

Zeta-potential is the electrostatic potential between the plane of shear (within the interfacial double layer) and the bulk fluid away from the interface. It is a very useful parameter for evaluating the stability of colloidal dispersion and the interactions among charged molecules. In this study, zeta potential was used in understanding the interactions between the negatively charged nanoparticles and the positively charged nisin molecules in the solution.

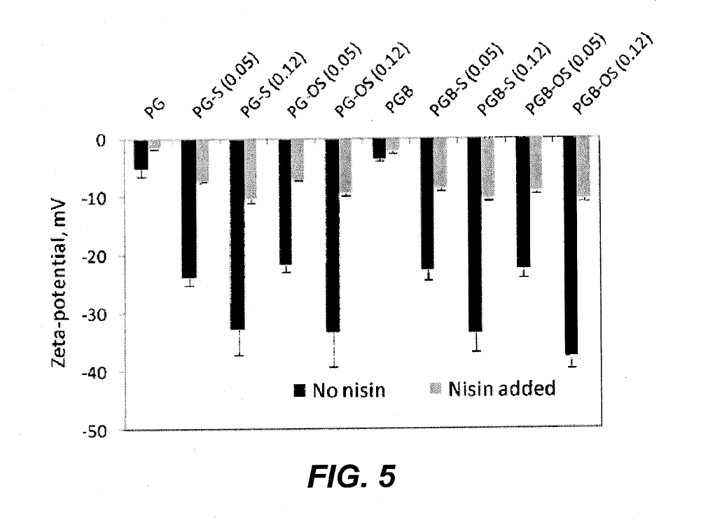

FIG. 5 shows the zeta-potentials of PG derivatives with and without added nisin. The zeta-potentials of PG and PGB at pH 5.5 were slightly negative (−5.1 for PG and −3.6 for PGB), suggesting the presence of a trivial amount of anionic compounds. Usually, purified PG contains approximately 1% protein, which could have contributed to the negative charges observed. With the grafting of succinate or octenyl succinate groups, the zeta-potential was substantially decreased (increased absolute value), indicating the negative charges introduced by carboxylate groups. In general, a DS of 0.05 led to a zeta-potential ranging from −22 to −24 mV, regardless of the involvement of PG, PGB, succinate, or octenyl succinate (FIG. 5). In contrast, a DS of 0.12 led to a zeta-potential around −33 to −38 mV for each type of PG derivative.

Adding 20 μg/mL nisin led to a significant increase (decrease in the absolute value) in zeta-potential for nanoparticles (FIG. 5). For PG-S (0.05), PG-OS (0.05), PGB-S (0.05), and PGB-OS (0.05), the addition of nisin changed the zeta-potential to −7.4, −7.1, −8.8, and −9.3 mV, respectively. For PG-S (0.12), PG-OS (0.12), PGB-S (0.12), and PB-OS (0.12), the addition of nisin changed the zeta-potential to −10.4, −9.4, −10.5, and −10.7 mV, respectively. Conceivably, the decrease in the absolute value of zeta-potential was due to the reduction in negative charge at the surface of nanoparticles caused by the adsorption of positively charged nisin molecules.

Nisin Loading to Nanoparticles

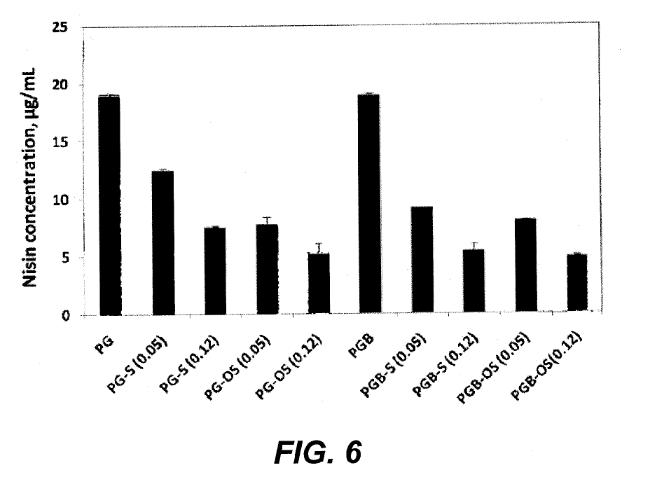

In this study, the loading of nisin to the nanoparticles was evaluated by measuring the concentration of nisin in the filtrate of ultrafiltration. The total nisin concentration in the original preparation was 20 μg/mL. For PG and PGB, the nisin concentration in the filtrate was 19 μg/mL (FIG. 6), suggesting negligible capability of non-substituted nanoparticles for loading nisin. For the substituted nanoparticles, their nisin-loading capability was affected by DS, substitution groups, and substrates. The impact of DS on nisin loading was the most evident. When DS increased from 0.05 to 0.12, the amount of non-loaded nisin was significantly reduced for all PG-S, PGOS, PGB-S, and PGB-OS nanoparticles. Considering the high zeta-potential absolute value associated with high DS (FIG. 5), it is believed that the electrostatic interaction between nanoparticles and nisin played an essential role in nisin adsorption.

At equivalent DS, octenyl succinate substitution usually led to a greater nisin loading than succinate. For example, the non-loaded nisin for PG-OS (0.05) (7.7 μg/mL) was lower than that for PG-S (0.05) (12.5 μg/mL), and 5.2 μg/mL for PG-OS (0.12) was lower than 7.5 μg/mL for PG-S (0.12). Therefore, in addition to the electrostatic interaction, the hydrophobic interaction between octenyl moieties and nisin also contributed to peptide adsorption.

The type of substrate (PG or PGB) also affected nisin loading. At equivalent DS, the amount of non-loaded nisin for PGB-S (0.05) was much lower than that for PG-S (0.05). Similar result was observed between PGB-S (0.12) and PG-S (0.12). In contrast, the differences between PG-OS and PGB-OS were less significant. As mentioned earlier, β-amylolysis has a thinning effect at the surface of nanoparticles (FIG. 4B) that may improve nisin loading. However, this effect seems to be interrelated with the type of substitution.

Initial Availability of Nisin Affected by PG Derivatives

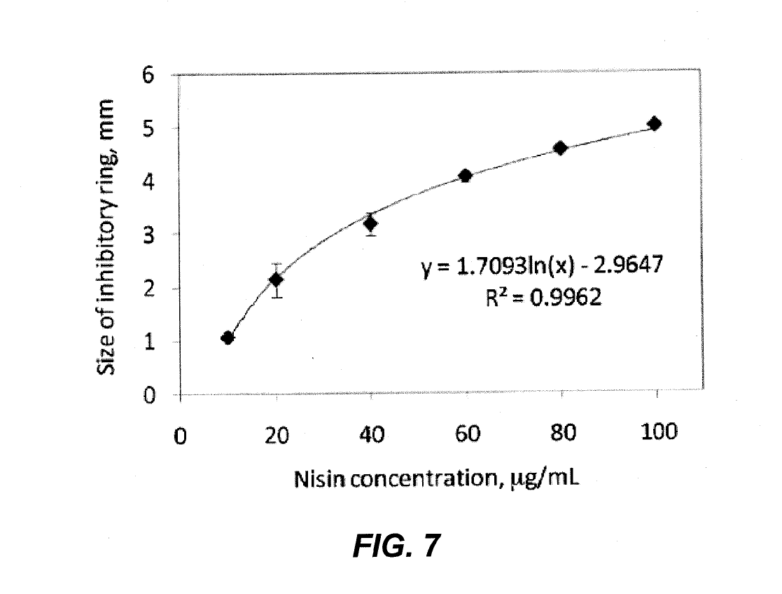

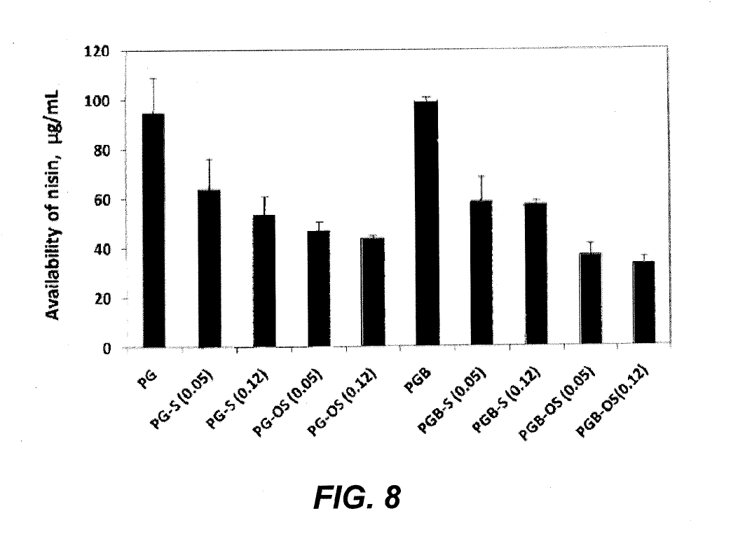

In this study, the inhibitory activity of nisin was evaluated using a diffusion test against L. monocytogenes. FIG. 7 shows the relationship between the size of inhibition ring and the concentration of free nisin. Using the equation shown in FIG. 7, the size of inhibition ring for each nisin preparation can be converted to the “availability of nisin”, i.e. the concentration of free nisin that offers the same inhibitory capability in the diffusion bioassay. FIG. 8 shows the initial availability of nisin for nanoparticle solutions containing 100 μg/mL nisin. For the non-substituted nanoparticles, PG and PGB, the initial availability of nisin was 94.8 and 99.4 μg/mL, respectively. This indicates that the initial inhibitory behavior of nisin in both PG and PGB solutions was essentially the same as that of the 100 μg/mL free nisin solution. In contrast, for PG derivatives, the initial availability of nisin was much lower than that for 100 μg/mL. For example, for PG-OS (0.12) and PGB-OS (0.12), the initial availability of nisin was 43.8 and 32.9 μg/mL, respectively. Evidently, the loading of nisin to nanoparticles was the primary factor in the reduction of the initial availability of nisin. In general, the availability of nisin was affected by OS, substitute groups, and substrates. Specifically, octenyl succinate substitution and β-amylolysis were more effective than high OS for reducing the initial availability of nisin.

Prolonged Nisin Efficacy

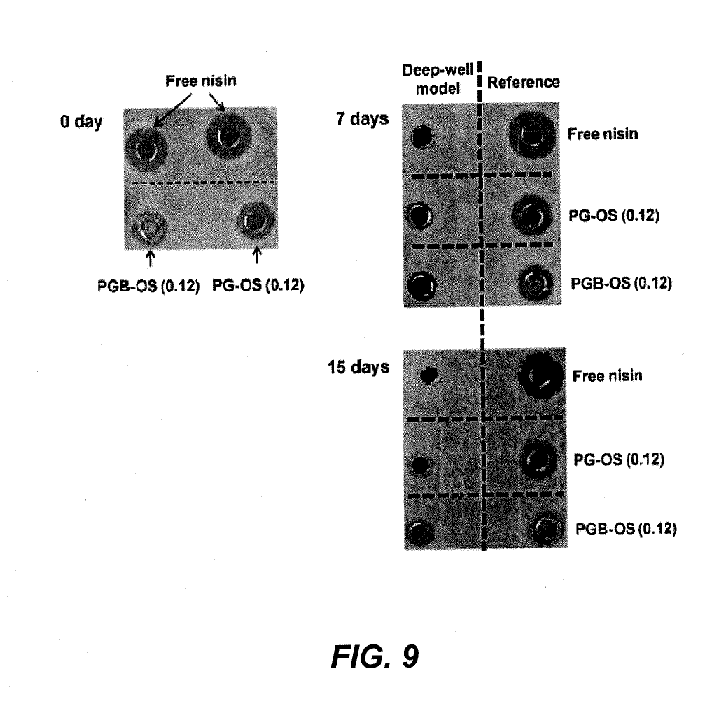

In this study, the residual activity of each nisin preparation stored in the BHI-agar deep-well model was evaluated using the size of inhibitory ring in the bioassay. FIG. 9 compares free nisin and preparations containing nisin and PG-OS (0.12) or PGB-OS (0.12) nanoparticles. For each preparation, inhibitory rings for both the model and reference are shown. For the reference groups, the size of inhibitory ring remained essentially the same over the 21-day storage, suggesting a high stability of nisin regardless of the presence of nanoparticles. Overall, the size of inhibitory ring was in the order of free nisin >PG-OS (0.12)>PGB-OS (0.12), reflecting the availability of nisin of individual preparations.

Deep-well model tests demonstrated the effectiveness of using nanoparticles to prolong the efficacy of nisin against L. monocytogenes. For the initial test at 0 day, the solution of free nisin showed the highest nisin activity. After 7 days, the activity of free nisin was negligible, whereas the activities of PG-OS (0.12) and PGB-OS (0.12) preparations were evident. After 15 days, the residual nisin activity was clearly retained for PGB-OS (0.12), whereas for PG-OS (0.12) the nisin activity was almost lost.

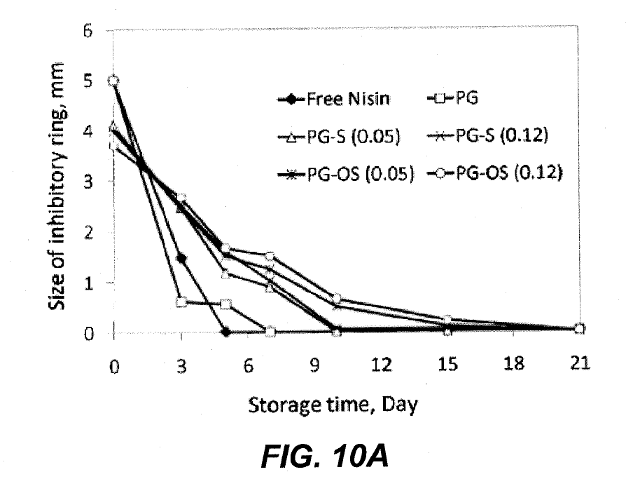

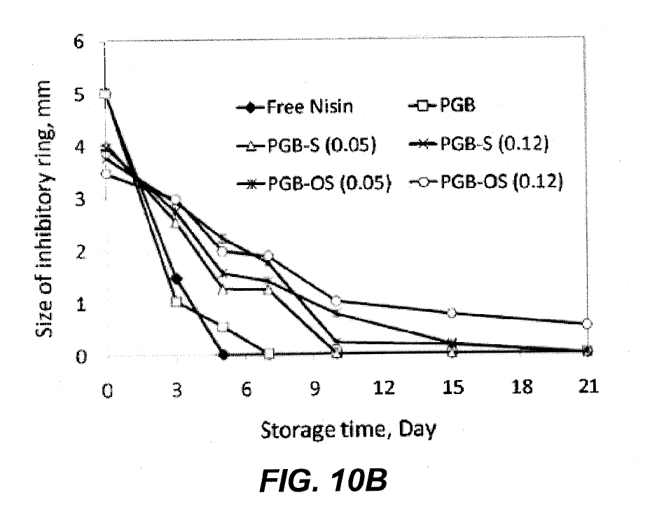

Antimicrobial activity during the 21-day storage at 4° C. is compared among various nisin preparations (FIGS. 10A-B). In general, nanoparticle-containing preparations showed reduced depletion of nisin activity compared to the solution of free nisin. The effect of PG and PGB was rather low, corresponding to their lack of capability to adsorb nisin (FIGS. 6 and 8). For substituted nanoparticles, octenyl succinate substitution correlated to a greater effect than succinate in reducing nisin depletion. This shows that the hydrophobic interaction played an important role in the retention of nisin activity, which was consistent with the high nisin loading (FIG. 6) and low initial availability of nisin (FIG. 8) associated with octenyl succinate substitution. In addition, PGB-based nanoparticles had a greater capability to retain nisin activity than did PG-based, regardless of the substitution with succinate or octenyl succinate. This shows the effect of nanoparticle surface structure on nisin loading and retention. Conceivably, the surface thinning of nanoparticles due to β-amylolysis resulted in a greater nisin loading, which led to a longer period of activity retention. Overall, DS, hydrophobicity, and glucan structure all affect nisin loading and release, and these factors can be used to design PG-based carbohydrate nanoparticles for prolonged efficacy of nisin.

Compared with film and liposome-based peptide carriers [10, 12-19], carbohydrate nanoparticles can be conveniently applied to target systems and easily manipulated for desirable loading and retention of antimicrobial peptide. Similar concepts have been proposed in drug delivery. For instance, nanoparticles made from poly(lactic-co-glycolic acid) (PLGA) were used to deliver anti-HIV-1 peptide [36]. However, nanocarriers used in drug delivery are mostly synthetic or inorganic, which are not suitable for food uses. In contrast, carbohydrate nanoparticles used in the current work are digestible [25] and abundant, showing potentials in both the food and drug areas.

Apart from the electrostatic interaction, the hydrophobicity of peptides is a major factor of loading and release. Recently, Bysell et al. [37] reported that by end-tagging antimicrobial peptides using oligotryptophan groups, the binding of peptides with poly(acrylic acid) microgels can be substantially improved. In our work, the superiority of PGB-OS with high DS value was related to the molecular characteristic of nisin, that is, the lysine residues offer positive charges and lanthionine and methyl lanthionine residues offer hydrophobicity [38]. In general, both electrostatic and hydrophobic interactions should be effectively utilized for designing carbohydrate nanocarriers of antimicrobial peptides.

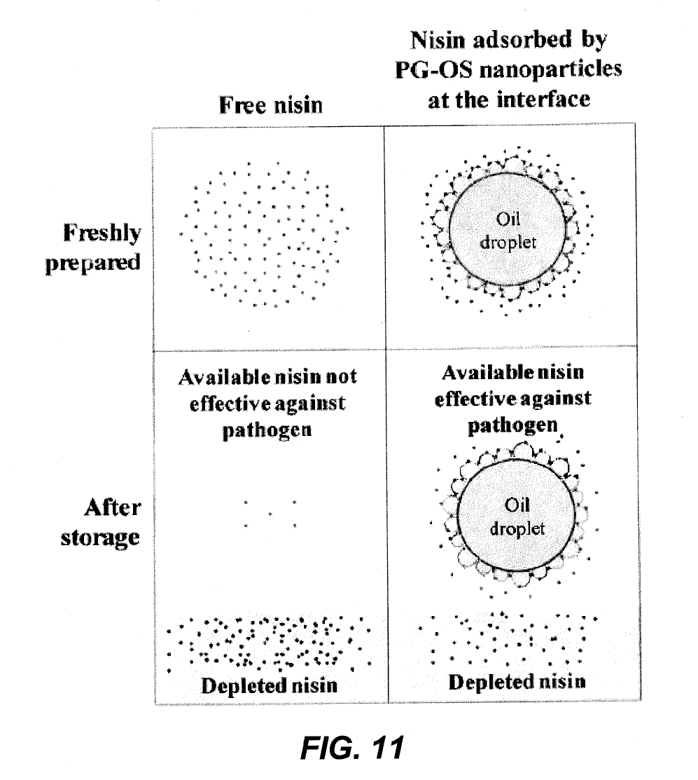

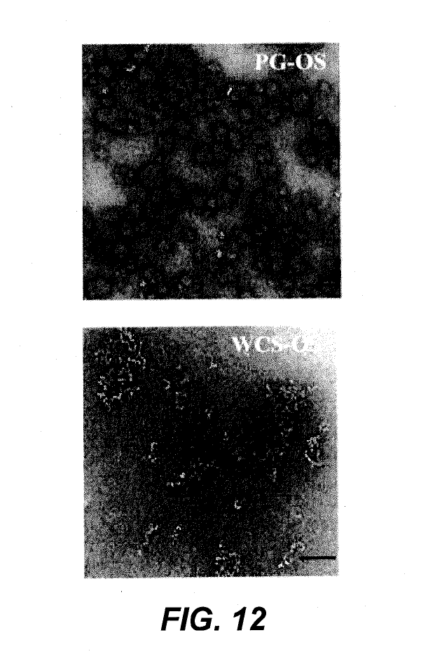

FIG. 11 conceptually depicts the adsorption of peptides at the interface of the PG-OS-stabilized oil droplets. This adsorption substantially reduces the number of free peptide molecules that are susceptible to quick depletion. To understand the impact of emulsifiers on the duration of peptide efficacy, in addition to PG-OS we selected two other amphiphilic materials: Tween 20, a small-molecule, neutral surfactant, and waxy corn starch octenyl succinate (WCS-OS), a hyperbranched carbohydrate polymer that can form stable emulsions. Both PG-OS and WCS-OS are amphiphilic, negatively charged macromolecules, but they have drastically different structures. The TEM images in FIG. 12 show PG-OS as dense nanoparticles and WCS-OS as highly dispersed, worm-like macromolecules.

Titration analysis showed that the degree of substitution (OS) was 0.013 for both PG-OS and WCS-OS. This is equivalent to an average of 13 octenyl succinate groups per 1,000 glucosyl units of PG or WCS. Analysis using high performance size-exclusion chromatography (HPSEC) combined with multi-angle laser light scattering (MALLS) indicated that the weight average molecular masses (M_{w}) of PG-OS and WCS-OS were 1.74±0.01×10⁷ and 2.31±0.14×10⁷ g/mol, respectively. The Z-average root mean square (RMS) radii (R_{z}) of PG-OS and WCS-OS were 25.83±0.31 and 115.70±4.10 nm, respectively. The dispersed molecular densities (defined as ρ=Mw/Rz³) (Wong et al., 2003) of PG-OS and WCS-OS were 1011.9 and 15.0 g/mol·nm³, respectively. This unusually high density of the PG-OS nanoparticles could lead to the formation of a thick, dense interfacial layer over the oil droplets in emulsions (FIG. 11).

Adsorption of PG-OS, WCS-OS, and Nisin in Emulsions

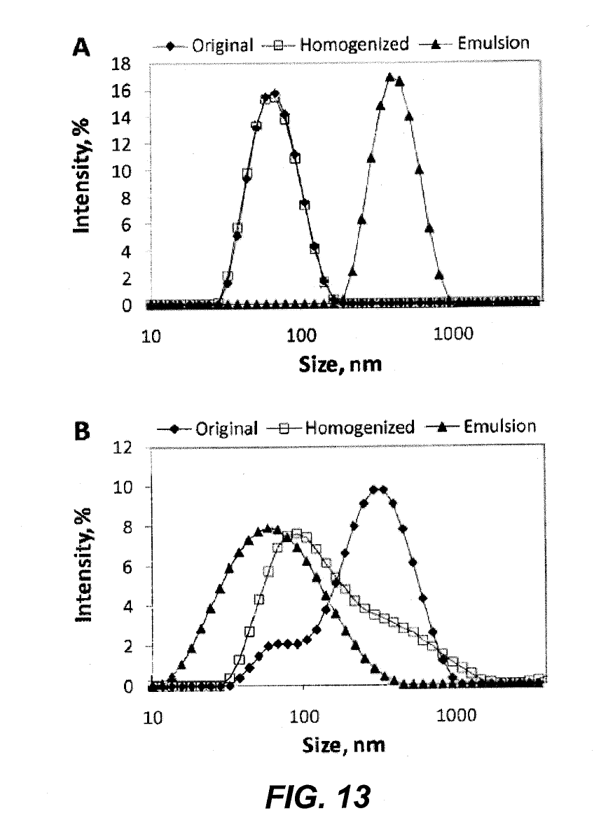

The particle size distribution of PG-OS, WCS-OS, and the emulsions stabilized by each was measured using dynamic light scattering. As shown in FIG. 13A, the hydrodynamic diameter (D_{H}) distribution of PG-OS was not affected by homogenization, showing resistance of the nanoparticles to the high shear induced by homogenization. In contrast, WCS-OS was rather fragile, being reflected by a substantial particle size reduction following homogenization (Z-average OH decreasing from 196 to 118 nm) (FIG. 13B).

In the presence of nisin, the Z-average D_{H} of droplets in the PG-OS-stabilized emulsion was 336 nm. Notably, free nanoparticles in the emulsion were undetectable, suggesting full adsorption of PG-OS at the oil-water interface (described in FIG. 11). In the WCS-OS-stabilized emulsion, the Z-average D_{H} of droplets was 50.2 nm, much lower than that of homogenized WCS-OS. This highlights the flexibility of homogenized WCSOS molecules to attach at the interface and assume a “shrunken” conformation to accommodate the nanoscale oil droplets.

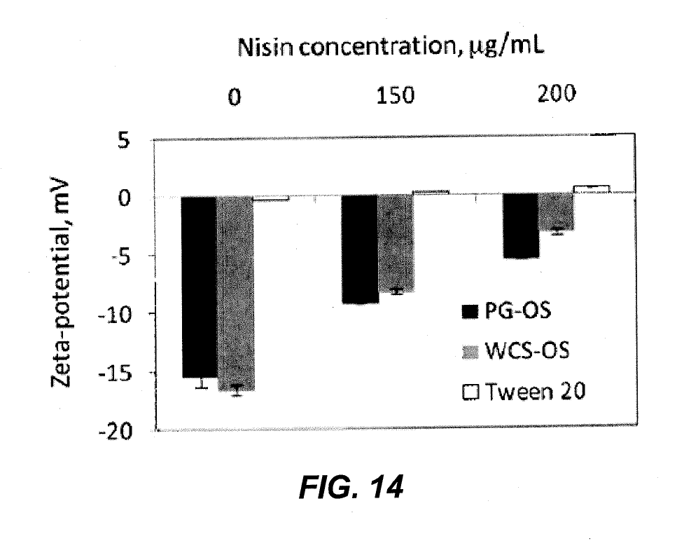

FIG. 14 shows the impact of nisin concentration on the zeta-potential of the emulsion droplets. Without nisin, the zeta-potentials of the PG-OS- and WCS-OS-stabilized emulsions were −15.5 and −16.7 mV, respectively. The addition of nisin substantially changed the zeta-potential for both the PG-OS and WCS-OS emulsions, and these changes were strongly related to the amount of nisin added. Evidently, the adsorption of nisin molecules occurred at the surface of the emulsion droplets. For the Tween 20-stabilized emulsion, the zeta-potential increased modestly from −0.3 to 0.6 mV with 200 μg/mL of nisin added, suggesting very low nisin adsorption at the surface of the oil droplets.

Antimicrobial Efficacy Against Listeria Monocytogenes During Extended Storage

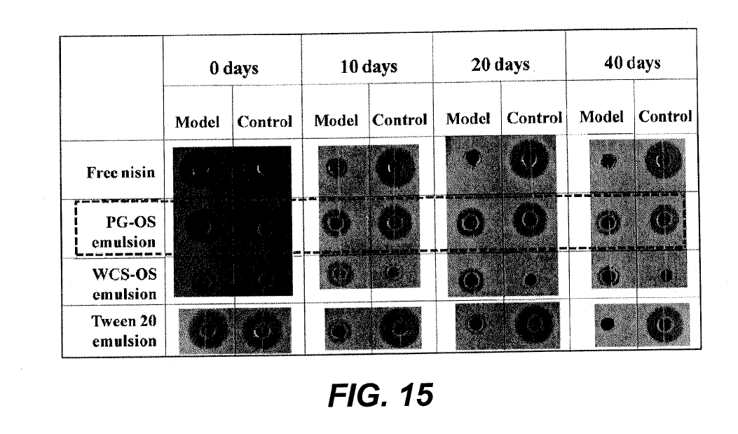

FIG. 15 shows the retention of nisin activity against L. monocytogenes indicated by the size of the inhibitory ring (defined in FIG. 1) during storage tests. For the “control” groups, nisin preparations were stored in regular test tubes at 4° C.; therefore, any change in ring size indicated a change in nisin availability caused only by the interaction between the peptide and the delivery system. As expected, the free nisin control did not show an appreciable change during storage, demonstrating the high stability of nisin. There was a minor reduction of ring size for both the PG-OS- and Tween 20-stabilized emulsions, indicating slowly increasing adsorption during storage. In contrast, the ring size of the WCS-OS-stabilized emulsion decreased rapidly to a negligible level after 10 days, which may have been caused by overly strong adsorption of peptide at the interface.

As shown in FIG. 15, the PG-OS-stabilized emulsion demonstrated the greatest ability to preserve nisin activity during extended storage. After 40 days, for the model groups the size of the inhibitory ring for the PG-OS emulsion was the largest among all preparations, whereas the rings for free nisin and the Tween 20 emulsion were undetectable. In fact, the ring size of the PG-OS emulsion at 40 days was larger than those of free nisin and the Tween 20 emulsion at 10 days. The ring size of the WCS-OS emulsion was always smaller than that of the PG-OS emulsion, particularly at 0, 20, and 40 days.

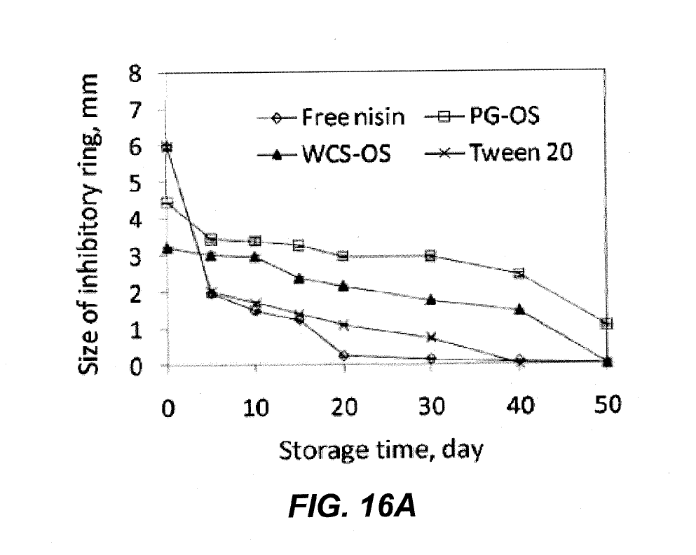

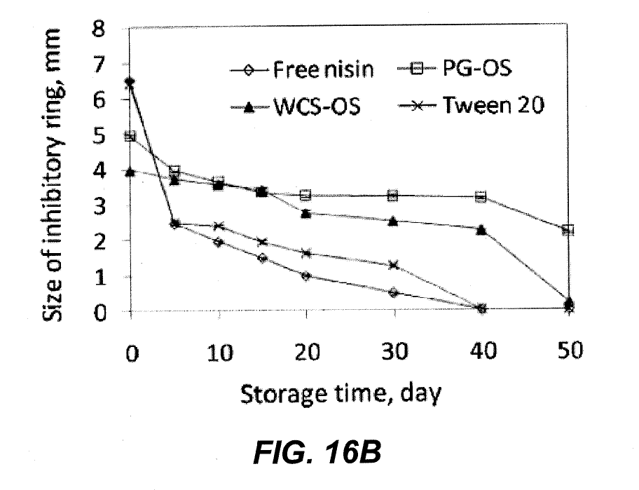

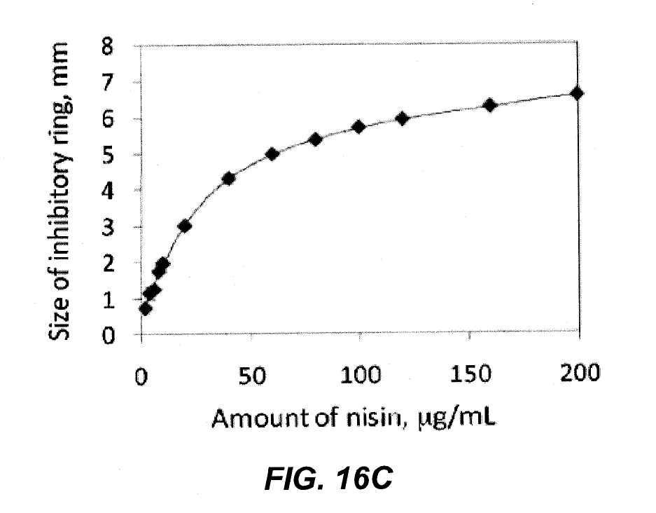

In FIGS. 16A and 16B, the preservation of nisin activity was quantitatively compared among the various preparations. Two initial total nisin concentrations, 150 and 200 μg/mL, were used in efficacy tests. A 6-order polynomial “standard curve” was also drawn (FIG. 16C) to correlate the size of the inhibitory ring with the amount of available nisin. In general, the initial activity of the free nisin preparation was the highest, but it decreased sharply in the first 5 days and continued to decrease in the later stages. With an initial nisin concentration of 150 μg/mL, the available nisin in the free nisin preparation was calculated to be 10.2 μg/mL after 5 days, <1 μg/mL after 20 days, and negligible after 40 days. In contrast, the PG-OS-stabilized emulsion showed substantial inhibitory effects during the extended storage period. Due to interfacial adsorption, the available nisin was initially about 43.8 μg/mL and then decreased slowly during storage to about 25.3 μg/mL after 5 days, 19.4 μg/mL after 20 days, and 14.3 μg/mL after 40 days.

The Tween 20-stablized emulsion showed minor improvement over free nisin after 10 days. Without wishing to be bound by a particular theory or to in any way limit the scope of the appended claims or their equivalents, it is presently believed that the hydrophobic interaction between nisin and the surface of the oil droplets may lead to a minor level of adsorption as indicated by the zeta-potential data (FIG. 14). This interaction, however, was apparently not sufficient to successfully prolong nisin efficacy.

The behavior of the WCS-OS-stabilized emulsion is noteworthy. Regardless of the initial amount of nisin, the initial ring size (and, accordingly, the amount of available nisin) was lower than that for the PG-OS-stabilized emulsion. This implies that nisin adsorption was stronger in the WCS-OS emulsion than in the PG-OS emulsion. Meanwhile, the retention of nisin activity observed for the WCS-OS emulsion was lower than that for the PG-OS emulsion throughout the storage period. The fact that nisin availability quickly declined in the control group (FIG. 15) but was somewhat retained in the model group suggests that certain components (possibly protein molecules) in the storage model may have reduced the over-adsorption of nisin at the interface of the droplets in the WCS-OS emulsion.

Potential in Food Applications

In the effort to retain the activity of antimicrobial compounds, most researchers have been focusing on the incorporation of active compounds to films. Specifically, these researches mimicked the scenario in which the bacterial contamination occurs before packaging, and the goals were to reduce the microbial growth during the storage of food in the initial package. The films have been prepared using either synthetic polymers such as plastics or biopolymers such as polysaccharides and proteins. For example, polyethylene film was used to retain nisin activity in the 20-day storage and a reduction from log₁₀6.3 to log₁₀3.6 was realized for B. thermosphacta (Siragusa et al., 1999). Nguyen et al. (2008) infused nisin into cellulose film to inhibit the growth of L. monocytogenes, achieving a 2 log CFU/g reduction compared with the non-nisin control after 14 days of storage. Nisin-containing polylactic acid films prepared by Jin and Zhang (2008) were tested against L. monocytogenes and a reduction of 4.5 log CFU/mL over the controls was shown. Similar studies have been conducted using films prepared from other materials, such as sodium caseinate (Kristo et al., 2008), soy protein (Sivarooban et al. 2008), alginate (Millette et al., 2007), and zein (Hoffman et al., 2001).

In most studies, the antimicrobial efficacy assay followed a procedure in which the food (or food model) were inoculated first with bacteria and then stored for a period of time during which the growth of bacteria was monitored. Conceivably, this procedure was used to test an effective release of antimicrobial compounds from films. In most antimicrobial studies, the control group did not contain antimicrobial compounds. In one report free nisin was used as the control (Millette et al., 2007), and the efficacy of nisin-containing alginate beads was shown to be lower than that of free nisin. This is not a surprise. With the same amount of nisin, the initial availability of peptide molecules of a free nisin preparation should be higher than that of a film. The comparison between free and film-incorporated nisin was not found in other publications, possibly due to an understanding that potential nisin depletion in food would justify the use of films for retaining nisin activity during storage.

Our study, on the other hand, directly compared the efficacy of nisin with and without a delivery system (e.g. PG-OS or WCS-OS emulsion). The design of our study is based on the concept that, a successful bacteriocin delivery system should not only show antimicrobial effect, but also show an effect greater than that of free nisin preparation. Another issue is the timing of bacterial contamination, and subsequent intended protection strategy. For L. monocytogenes, it was recently identified that the contamination at the production and packaging stages has been largely under control, while 80% of Listeria-related deaths are attributed to ready-to-eat foods from deli counters (Houchins, 2008). Therefore, it is important to prevent bacterial growth after the exposure of food to environment at retail and home storages. One approach to address this challenge is to apply antimicrobial compounds at the packaging stage and maintain the antimicrobial activity throughout the life of this product. Another approach is to apply antimicrobial compounds right after opening the package. Both approaches should be able to keep the food protected from bacterial contamination before consumption.

From this perspective, this study was undertaken for developing a strategy to protect food from potential contamination in the later (rather than earlier) stages of product life. Using PG-OS emulsion as a delivery system, the nisin efficacy against Listeria can be retained for as long as 50 days. The established strategy is novel, not only due to the use of carbohydrate nanoparticle-mediated assemblies, but also due to its potential to reduce a recently identified but major cause of pathogenic contamination. One advantage of using a dispersion system (over a film-based strategy) to deliver bacteriocin is to retain the majority of antimicrobial compounds with food after removing the package.

The PG-OS-stabilized emulsion showed an outstanding ability to prolong the efficacy of bacteriocin nisin against the food pathogen L. monocytogenes. The use of amphiphilic carbohydrate nanoparticle-mediated colloidal assembly for prolonged delivery of bioactive compounds has not been reported previously. A key finding of this work is that the interface of carbohydrate nanoparticle-stabilized emulsion can be used to deliver bioactive compounds. PG-OS is a novel, digestible nanomaterial, and its potential benefits are beginning to be revealed (Scheffler et al., 2010a, b). Importantly, the structure of phytoglycogen nanoparticles can be manipulated through biological, chemical, and enzymatic approaches, allowing the creation of a new class of nano-constructs and devices. This study was conducted from the perspective of food applications; however, the methodology established can be broadly used in various biological and physiological systems.

The following literature provides information that may be useful in accordance with the present teachings and each document is hereby incorporated by reference in its entirety, except that in the event of any inconsistent disclosure or definition from the present specification, the disclosure or definition herein shall be deemed to prevail:
(1) J. H. Goff, A. K. Bhunia, M. G. Johnson, Complete inhibition of low levels of Listeria monocytogenes on refrigerated chicken meat with pediocin AcH bound to heat-killed Pediococcus acidilactici cells, J. Food Prot. 59 (1996) 1187-1192.
(2) Food Safety and Inspection Service, 2008. Recall Information Center. http://www.fsis.usda.gov/fsis_recalis/Recall_Case_Archive_{—}2008/index.asp.
(3) Food Safety and Inspection Service, 2007. Recall Information Center. http://www.fsis.usda.gov/fsis_recalis/Recall_Case_Archive_{—}2007/index.asp.
(4) W. Liu, J. N. Hansen, Some chemical and physical properties of nisin, a small-protein antibiotic produced by Lactococcus lactis, Appl. Environ. Microbiol. 56 (1990) 2551-2558.
(5) B. B. Bonev, W. C. Chan, B. W. Bycroft, G. C. K. Roberts, A. Watts, Interaction of the lantibiotic nisin with mixed lipid bilayers: A P-31 and H-2 NMR study, Biochem. 39 (2000) 11425-11433.
(6) E. Breukink, C. vanKraaij, R. A. Demel, R. J. Siezen, O. P. Kuipers, B. de Kruijff, The C-terminal region of nisin is responsible for the initial interaction of nisin with the target membrane, Biochem. 36 (1997) 6968-6976.
(7) E. Breukink, P. Ganz, B. de Kruijff, J. Seelig, Binding of nisin Z to bilayer vesicles as determined with isothermal titration calorimetry, Biochem. 39 (2000) 10247-10254.
(8) E. Breukink, B. de Kruijff, Lipid II as a target for antibiotics, Nat. Rev. Drug Discov. 5 (2006) 321-332.
(9) J. Delves-Broughton, Nisin as a food preservative, Food Australia. 57 (2005) 525-527.
(10) S. Quintavalla, L. Vicini, Antimicrobial food packaging in meat industry, Meat Sci. 62 (2002) 373-380.
(11) N. L. Rose, P. Sporns, M. E. Stiles, L. M. McMullen, Inactivation of nisin by glutathione in fresh meat, J. Food Sci. 64 (1999) 759-762.
(12) T. Padgett, I. Y. Han, P. L. Dawson, Incorporation of food-grade antimicrobial compounds into biodegradable packaging films, J. Food Prot. 61 (1998) 1330-1335.
(13) G. R Siragusa, C. N. Cutter, J. L. Willett, Incorporation of bacteriocin in plastic retains activity and inhibits surface growth of bacteria on meat, Food Microbiol. 16 (1999) 229-235.
(14) R. D. Joerger, Antimicrobial films for food applications: a quantitative analysis of their effectiveness, Packaging Tech. & Sci. 20 (2007) 231-273.
(15) H. Neetoo, M. Ye, H. Chen, Effectiveness and stability of plastic films coated with nisin for inhibition of Listeria monocytogenes, J. Food Prot. 70 (2007) 1267-1271.
(16) R Laridi, E. E. Kheadr, R. O. Benech, J. C. Vuillemard, C. Lacroix, I. Fliss, Liposome encapsulated nisin Z: optimization, stability and release during milk fermentation, Int. Dairy J. 13 (2003) 325-336.
(17) R. O. Benech, E. E. Kheadr, C. Lacroix, I. Fliss, Impact of nisin producing culture and liposome-encapsulated nisin on ripening of Lactobacillus added-Cheddar cheese, J. Dairy Sci. 86 (2003) 1895-1909.
(18) L. M. Were, B. D. Bruce, P. M. Davidson, J. Weiss, Size, stability, and entrapment efficiency of phospholipid nanocapsules containing polypeptide antimicrobials, J. Agric. & Food Chem. 51 (2003) 8073-8079.
(19) M. Taylor, S. Gaysinsky, P. M. Davidson, B. Bruce, J. Weiss, Characterization of antimicrobial-bearing liposomes by zeta-potential, vesicle size, and encapsulation efficiency, Food Biophys. 2 (2007) 1-9.
(20) M. G. James, D. S. Robertson, A. M. Myers, Characterization of the maize gene sugary1, a determinant of starch composition in kernels, Plant Cell. 7 (1995) 417-429.
(21) Y. Yao, Chapter: Biosynthesis of starch, in Comprehensive Glycoscience edited by Hans Kamerling, Elsevier, Amsterdam, The Netherlands, 2007.
(22) A. M. Myers, M. K. Morell, M. G. James, S. G Ball, Recent progress toward understanding the amylopectin crystal, Plant Physiol. 122 (2000) 989-997.
(23) Y. Nakamura, Towards a better understanding of the metabolic system for amylopectin biosynthesis in plants: Rice endosperm as a model tissue. Plant & Cell Physiol. 43 (2002) 718-725.
(24) S. L. Scheffler, X. Wang, L. Huang, F. San-Martin Gonzalez, Y. Yao, Phytoglycogen octenyl succinate, an amphiphilic carbohydrate nanoparticle, and e-polylysine to improve lipid oxidative stability of emulsions. J. Agric. & Food Chem. 58 (2010) 660-667.
(25) S. L. Scheffler, L. Huang, L. Bi, Y. Yao, In vitro digestibility and emulsification properties of phytoglycogen octenyl succinate. J. Agric. & Food Chem. 58 (2010) 5140-5146.
(26) O. B. Wurzburg, Modified Starch, in Food Polysaccharides and Their Applications edited by A. M. Stephen, G. O. Phillips and P. A Williams, second ed., CRC, Boca Raton, Fla., 2006.
(27) D. S. Pisal, M. P. Kosloski, S. V. Balu-lyer, Delivery of Therapeutic Proteins, J. Pharm. Sci. 99 (2010) 2557-2575.
(28) C. J. Cui, V. C. Stevens, S. P. Schwendeman S P, Injectable polymer microspheres enhance immunogenicity of a contraceptive peptide vaccine, Vaccine 25 (2007) 500-509.
(29) A. M. Sophocleous, Y. Zhang, S. P. Schwendeman, A new class of inhibitors of peptide sorption and acsylation in PLGA, J. Control. Release 137 (2009) 179-184.
(30) R. Sheshala, K. K Peh, Y. Darwis, Preparation, characterization, and in vivo evaluation of insulin-loaded PLAPEG microspheres for controlled parenteral drug delivery, Drug Dev. Ind. Pharm. 35 (2009) 1364-1374.
(31) J. Ostergaard, E. H. Moeller, Ghrelin-liposome interactions: Characterization of liposomal formulations of an acylated 28-amino acid peptide using CE, Electrophoresis 31 (2010) 339-345.
(32) F. D. Hu, Y. Hong, H. Yuan, Preparation and characterization of solid lipid nanoparticles containing peptide, Int. J. Pharm. 273 (2004) 29-35.
(33) Y. P. Chan, R. Meyrueix, R. Kravtzoff, F. Nicolas, K. Lundstrom, Review on Medusa (R): a polymer-based sustained release technology for protein and peptide drugs, Expert Opin. Drug Deliv. 4 (2007) 441-451.
(34) X. J. Gao, X. E. Zhang, Z. M. Wu, X. J. Zhang, Z. Wang, C. X. Li, Synthesis and physicochemical characterization of a novel amphiphilic polylactic acid-hyperbranched polyglycerol conjugate for protein delivery, J. Control. Release 140 (2009) 141-147.
(35) J. Shin, S. Simsek, B. L. Reuhs, Y. Yao, Glucose release of water-soluble starch-related a-glucans by pancreatin and amyloglucosidase is affected by the abundance of a-1,6-glucosidic linkages. J. Agric. & Food Chem. 56 (2008) 10879-10886.
(36) A. S. Ham, M. R. Cost, A. B. Sassi, C. S. Dezzutti, L. C. Rohan, Targeted delivery of PSC RANTES for HIV-1 prevention using biodegradable nanoparticles. Pharm. Res. 26 (2009) 502-511.
(37) H. Bysell, P. Ransson, A Schmidtchen, M. Malmsten, Effect of hydrophobicity on the interaction between antimicrobial peptides and poly(acrylic acid) microgels. J. Phys. Chem. B 114 (2010) 1307-1313.
(38) A. Peschel, H. G. Sahl, The co-evolution of host cationic antimicrobial peptides and microbial resistance. Nat. Rev. Microbiol. 4 (2006) 529-536.

The following literature provides additional information that may also be useful in accordance with the present teachings and each document is hereby incorporated by reference in its entirety, except that in the event of any inconsistent disclosure or definition from the present specification, the disclosure or definition herein shall be deemed to prevail:
(1) Aveyard R., Binks B. P., Clint J. H. 2003. Emulsions stabilised solely by colloidal particles. Adv. Colloid Interface Sci. 100:503-546.
(2) Binks B. P., Desforges A., Duff D. G. 2007. Synergistic stabilization of emulsions by a mixture of surface-active nanoparticles and surfactant. Langmuir 23:1098-1106.
(3) Dinsmore A. D., Hsu M. F., Nikolaides M. G., Marquez M., Bausch A. R., Weitz D. A. 2002. Colloidosomes: selectively permeable capsules composed of colloidal particles. Science 298:1006-1009.
(4) Hoffman K. L., Han I. Y., Dawson P. L. 2001 Antimicrobial effects of corn zein films impregnated with nisin, lauric acid, and EDTA. J. Food Protect. 64:885-889.
(5) Gallagher D. L., Ebel E. D., Kause J. R. 2003a. Executive summary 2003 FSIS Listeria risk assessment (http://www.fsis.usda.gov/PDF/Lm_DelLRisk_Assess_ExecSumm_{—}2003.pdf).
(6) Gallagher D. L., Ebel E. D., Kause J. R. 2003b. FSIS risk assessment for Listeria monocytogenes in deli meats (http://www.fsis.usda.gov/OPPDE/RDAD/FRPubs/97013F/ListeriaReport.pdf).
(7) Houchins J. 2008. Listeria control in consumer protein products. 1 FT Newsletter (http://www.am-fe.ift.org/cmsl?pid=1000750).
(8) Jin T., Zhang H. 2008. Biodegradable polylactic acid polymer with nisin for use in antimicrobial food packaging. J. Food Sci. 73:M127-M134.
(9) Kristo E., Koutsoumanis K. P., Biliaderis C. G. 2008. Thermal, mechanical and water vapor barrier properties of sodium caseinate films containing antimicrobials and their inhibitory action on Listeria monocytogenes. Food Hydrocolloid. 22:373-386.
(10) Millette M., Le Tien C., Smoragiewicz W., Lacroix M. 2007. Inhibition of Staphylococcus aureus on beef by nisin-containing modified alginate films and beads. Food Control. 18:878-884.
(11) Neetoo H., Ye M., Chen H. 2007. Effectiveness and stability of plastic films coated with nisin for inhibition of Listeria monocytogenes. J. Food Prot. 70:1267-1271.
(12) Nguyen V. T., Gidley M. J., Dykes G. A. 2008. Potential of a nisin-containing bacterial cellulose film to inhibit Listeria monocytogenes on processed meats. Food Microbiol. 25:471478.
(13) Pickering S. U. 1907. Emulsions. J. Chem. Soc. 91:2001-2021.
(14) Pongtharangkul, T., Demirci, A. 2004. Evaluation of agar diffusion bioassay for nisin quantification. Appl. Microbiol. Biotechnol. 65:268-272.
(15) Rossier-Miranda F. J., Schroen CGPH, Boom R. M. 2009. Colloidosomes: Versatile microcapsules in perspective. Colloids Surf., A 343:43-49.
(16) Sivarooban T., Hettiarachchy N. S., Johnson M. G. 2008. Physical and antimicrobial properties of grape seed extract, nisin, and EDTA incorporated soy protein edible films. Food Res. Int. 41:781-785.
(17) Velev O. D., Furusawa K., Nagayama K. 1996. Assembly of latex particles by using emulsion droplets as templates 1. Microstructured hollow spheres. Langmuir 12:2374-2384.
(18) Wang D. Y., Duan H. W., Mohwald H. 2005. The water/oil interface: the emerging horizon for self-assembly of nanoparticles. Soft Matter 1:412-416.
(19) Wong K., Kubo A., Jane J., Harada K., Satoh H., Nakamura Y. 2003. Structures and properties of amylopectin and phytoglycogen in the endosperm of sugary-1 mutants of rice. J. Cereal Sci. 37:139-149.

The foregoing detailed description and accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

1-24. (canceled)

25. A nanoparticle comprising:
a carbohydrate carrier; and
a bacteriocin.

26. The nanoparticle of claim 25, wherein the bacteriocin comprises a peptide bacteriocin.

27. The nanoparticle of claim 25, wherein the bacteriocin comprises a lantibiotic.

28. The nanoparticle of claim 27, wherein the lantibiotic is selected from the group consisting of nisin, epidermin, subtilin, and combinations thereof.

29. The nanoparticle of claim 27, wherein the lantibiotic comprises nisin.

30. The nanoparticle of claim 25, wherein the carbohydrate carrier comprises an anhydride-modified phytoglycogen or glycogen-type material.

31. The nanoparticle of claim 30, wherein the anhydride comprises succinic anhydride.

32. The nanoparticle of claim 30, wherein the anhydride comprises octenyl succinic anhydride.

33. The nanoparticle of claim 25 wherein the carbohydrate carrier comprises phytoglycogen octenyl succinate.

34. The nanoparticle of claim 25, wherein the carbohydrate carrier comprises phytoglycogen β-dextrin or an anhydride-modified phytoglycogen β-dextrin.

35. A method for prolonging efficacy of a bacteriocin against a food pathogen comprising:
providing the bacteriocin in a delivery system; and
inhibiting the food pathogen by the bacteriocin;
wherein a duration of efficacy of the bacteriocin against the food pathogen when the bacteriocin is provided in the delivery system exceeds a duration of efficacy of the bacteriocin when the bacteriocin is provided without the delivery system.

36. The method of claim 35, wherein the delivery system is emulsion-based.

37. The method of claim 36, wherein the emulsion comprises a negative surface charge.

38. The method of claim 36, wherein the emulsion comprises carbohydrate nanoparticles.

39. The method of claim 35, wherein the bacteriocin comprises a peptide bacteriocin.

40. The method of claim 35, wherein the bacteriocin comprises a lantibiotic.

41. The method of claim 40, wherein the lantibiotic is selected from the group consisting of nisin, epidermin, subtilin, and combinations thereof.

42. The method of claim 40, wherein the lantibiotic comprises nisin.

43. The method of claim 38, wherein the carbohydrate comprises an anhydride-modified phytoglycogen or glycogen-type material.

44. The method of claim 43, wherein the anhydride comprises succinic anhydride.

45. The method of claim 43, wherein the anhydride comprises octenyl succinic anhydride.

46. The method of claim 38, wherein the carbohydrate comprises phytoglycogen octenyl succinate.

47. The method of claim 38, wherein the carbohydrate comprises phytoglycogen β-dextrin or an anhydride-modified phytoglycogen β-dextrin.

48. The method of claim 35, wherein the food pathogen comprises Listeria monocytogenes.
